(19) United States
(12) Patent Application Publication
(10) Pub. No.: US 2022/0015813 A1
(43) Pub. Date: Jan. 20, 2022

(54) Medical Fastener or Device with Dual Surface Topography

(71) Applicants: Nicholas Cordaro, Encinitas, CA (US); Andres Talamantes, Vista, CA (US)

(72) Inventors: Nicholas Cordaro, Encinitas, CA (US); Andres Talamantes, Vista, CA (US)

(21) Appl. No.: 17/380,261

(22) Filed: Jul. 20, 2021

Related U.S. Application Data
(60) Provisional application No. 63/053,791, filed on Jul. 20, 2020.

(51) Int. Cl. A61B 17/86; A61B 90/98

(52) U.S. Cl. CPC A61B 17/8625; A61B 2017/00526; A61B 90/98

(57) ABSTRACT

An improved bone screw or medical device comprising at least one smooth surface and at least one textured surface. The improved bone screw has a cylindrical body with a first end and a dosed second end. The head is positioned at the first end and configured to engage with a driver to advance the bone screw into a bane, and a tip is positioned at die second end. The exterior thread of the screw component has a smooth surface and a textured surface on at least one side and, preferably, only one side. The textured surface promotes honey on-growth and mechanical interlocking, and prevents premature withdrav,u1 of the bone screw from the bone. The smooth surface reduces the amount of insertion torque required during the installation process.

### CROSS-REFERENCE TO RELATED APPLICATION

The present application claims priority to and the benefit of U.S. Provisional Application No. 63/053,791 filed on Jul. 20, 2020, which is incorporated herein by reference in its entirety.

### FIELD OF THE INVENTION

The present invention relates generally to a medical device such as an implant, plate, rod or screw with a dual texture or other surface topography. More specifically, the dual textured surface of the medical device provides for mechanical interlocking and boney on-growth while in vivo. By providing the unique surface topography, back out or release of the screw or other medical implant from the bone is restricted or reduced. Accordingly, the present specification makes specific reference thereto. However, it is to be appreciated that aspects of the present invention are also equally amenable to other like applications, devices and methods of manufacture.

### BACKGROUND OF THE INVENTION

By way of background, various types of fasteners, such as surgical screws, pins and the like are used to engage implants, such as rods, screws and other devices to bone. In the spinal field, bone screws and pins are commonly used to attach plates, rods and other types of implants and devices to one or more vertebrae. Specifically, bone screws or pins are often used in orthopedic surgery to secure bone sections to each other or to artificial joints, plates, implants or other structural members to be retained in place to allow the bone time to grow into the screw, fastener or implant. The bone screws, which usually have a head for receiving a driver tool, and a threaded shank portion, are threaded or screwed into the bone to secure tendons, medical appliances and the like to the bone.

Additionally, compressive plating is a well-known technique in the medical field for imparting the stabilization needed or that is desirable for proper healing of the bone after surgery. In a compressive plating procedure, a rigid, typically metal plate is placed on the outer surface of the bone across the fracture area, and bone screws or other fasteners extend through the plate and are secured into the bone on either side of the fracture in a mariner which permits the rigid plate to offer support to the bone during the healing process. The bone screws include a threaded portion which is positioned along the shaft and which are adapted to engage the patient's cortical bone material. The threaded portion grips the area of the bone surrounding the area where the screw is inserted. Most commonly, the head portion of the bone screw has a standard screw-head configuration which provides sufficient compressive force about a corresponding round screw hole of the plate as the bone screw is threaded into the patient's bone or tendon, thereby causing compression of the plate against the bone and or tendon and the resulting desired stabilization and securement of the plate in the area of the fracture.

However, one existing limitation to compressive plating is the potential for post-operative micro-motion between the bone screw and the bone. More specifically, as the patient becomes mobile post-operatively and resumes more typical daily activities, the portion of the bone screw within the bone can begin to withdraw, wobble or loosen, thereby resulting in one or more of the following undesirable consequences: (a) loosening of the entire compression plate assembly; (b) diminishing the stability of the set fracture configuration; and/or (c) a loss of compression across the fracture area. Further, this micro-motion can also cause complications for the patient during recovery and may result in additional surgery to correct the loosening of the plate and/or implant.

Consequently, there exists a long felt need in the art for a medical device, such as an improved bone screw, that comprises a surface topography for allowing honey on-growth to the screw or fastener and mechanical interlocking of the improved bone screw and other implants to prevent screw loosening, back out or withdrawal, particularly in a compressive plating setting. There is also a long felt need in the art for an improved bone screw or fastener that not only reduces the potential likelihood of micro-motion or loosening after insertion, but also a fastening system that decreases, or does not unduly increase, the amount of torque needed to install the bone screw or fastener into the patient's bone. Finally, there is also a long felt need in the art for a bone screw or other fastener that is relatively inexpensive to manufacture and that is both safe and easy to use, and avoids the shortcomings of prior art medical-type fasteners.

More specifically, the present invention discloses an improved medical device such as a bone screw, fastener, or implant that comprises at least one smooth surface for decreased insertion torque, and at least one textured surface to promote boney on-growth and mechanical interlocking. While this specification makes specific reference to an improved bone screw and the various screw components of the present invention as a way in which to prevent unwanted bone screw back out, loosening or withdrawal, it should be appreciated by those of ordinary skill in the art that aspects of the present invention are also equally amenable to other like applications, and other such medical devices and/or back out issues.

### SUMMARY OF THE INVENTION

The following presents a simplified summary in order to provide a basic understanding of some aspects of the disclosed innovation. This summary is not an extensive overview, and it is not intended to identify key or critical elements or to delineate the scope thereof. Its sole purpose is to present some concepts in a simplified form as a prelude to the more detailed description that is presented later.

The subject matter disclosed and claimed herein, in one aspect thereof, comprises a bone screw or medical fastener that has at least one smooth surface for ease of insertion of the bone screw or fastener into the patient's bone and or surrounding tendons, and at least one textured surface or other surface topography for mechanical interlocking and to promote boney on-growth of the bone to the medical fastener. More specifically, the screw component comprises a generally cylindrical body having a first end and a closed second end. The cylindrical body further includes an exterior thread on an exterior cylindrical surface portion of the cylindrical body, a head at the first end configured to engage with a driver or other insertion tool to advance or position the bone screw into the bone and a penetrating tip at the second end. The cylindrical body, the head and the tip are preferably formed of a single unitary construction. Further, the surface of the exterior thread would have a controlled textured surface in one direction only, that is the material which is coated, grown on or otherwise provided on the surface to create the particular topography has a single orientation extending generally upwardly from the surface of the medical fastener.

In an alternative embodiment of the present invention, the improved bone screw has a screw component that has a smooth surface on the +/−X, +/−Y, and +Z directions of the exterior thread, and a textured surface on the −Z direction of the exterior threaded portion. More specifically, the smooth surfaces of the screw component allow the screw to be inserted into the patient's bone relatively easily by minimizing the amount of insertion torque required for installation, and the textured surface of the negative side (−Z direction) makes it more difficult for the screw component to come out, loosen or withdraw from the patient's bone prematurely. That is the surface topography is provided in the opposite direction of insertion.

In another embodiment of the present invention, the medical device can be an implant or other stabilizing element component wherein all surfaces of the implant component are relatively smooth, with the exception of the bone facing surfaces. In this embodiment, the bone facing surfaces of the implant component would have a textured surface to promote Wiley on-growth to fuse the implant or other stabilizing element with the bone.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and is intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a perspective view of the various machine parameters that may be used to create a textured surface on one potential embodiment of the bone screw component of the present invention in accordance with the disclosed architecture;

FIG. 2 illustrates a perspective view of the scan images of the hatch intersections, as separated by the stripe distance on one potential embodiment of the bone screw component of the present invention in accordance with the disclosed architecture;

FIG. 3 illustrates a perspective view of the scan images of the contour layers and hatch lines of one potential embodiment of the bone screw component of the present invention in accordance in accordance with the disclosed architecture;

FIG. 4A illustrates a perspective view of one potential embodiment of the bone screw component of the present invention in accordance with the disclosed architecture, wherein the bone screw has a textured surface on its exterior threads;

FIG. 4B illustrates a perspective view of one potential embodiment of the bone screw component of the present invention in accordance with the disclosed architecture, wherein the bone screw further comprises an RFID chip;

FIG. 4C illustrates a perspective view of one potential embodiment of the bone screw component of the present invention in accordance with the disclosed architecture, wherein the bone screw is inserted into a plate having a textured surface on only one side thereof;

FIG. 5 illustrates a perspective close-up view of the textured surface on the exterior thread of one potential embodiment of the bone screw component of the present invention in accordance with the disclosed architecture; and

FIG. 6 illustrates a block diagram of one potential embodiment of the method of manufacturing the bone screw component of the present invention in accordance with the disclosed architecture.

### DETAILED DESCRIPTION OF THE INVENTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof.

As noted above, there is a long felt need in the art for a medical device, such as an improved bone screw or fastener, that comprises a surface topography for allowing honey on-growth to the screw or fastener and mechanical interlocking of the improved bone screw and other implants to prevent screw loosening, back out or withdrawal, particularly in a compressive plating setting. Moreover, there is also a long felt need in the art for an improved bone screw or fastener that not only reduces the potential likelihood of micro-motion or loosening after insertion, but that also provides a fastening system that decreases, or does not unduly increase, the amount of torque needed to install the bone screw or fastener into the patient's bone. Finally, there is also a long felt need in the art for a bone screw or other fastener that is relatively inexpensive to manufacture and that is both safe and easy to use, and avoids the shortcomings of prior art medical-type fasteners.

Generally stated, and in one embodiment thereof, the present invention discloses an improved bone screw or other medical fastener having a threaded component that is comprised of at least one smooth surface and at least one textured surface. More specifically, the improved bone screw or medical fastener comprises a generally cylindrical body having threading thereon extending radially outwardly from a central core or shaft and a first end and a closed second end. An engagement configuration provided on a head is positioned at the first end of the generally cylindrical body and is designed to engage with a driver or other insertion tool to advance the improved bone screw or medical fastener into a patient's bone. A penetrating tip is positioned at the opposing second end from the head or first end. Further, the threading on the exterior of the cylindrical body further comprises a textured surface or other surface topography on at least one side or in at least one direction. The textured surface promotes boney on-growth and mechanical interlocking between the fastener and the bone into which it has been inserted. In an alternative embodiment, the present medical device invention may include an implant component, such as a rod or plate wherein all surfaces of the implant are smooth, except for the bone facing or engaging surfaces. More specifically, the bone facing surfaces would have a textured surface or other surface topography to promote boney on-growth and mechanical interlocking to prevent the loosening of the fastener and the bone once the fastener is inserted into the bone.

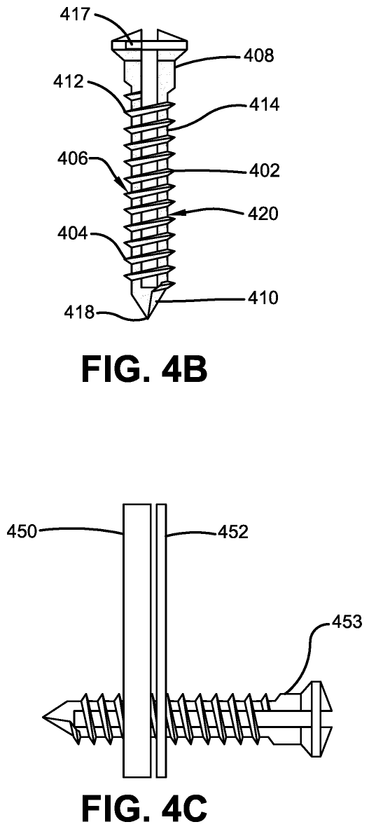

Referring initially to the drawings, FIG. 4A illustrates one possible embodiment of the improved bone screw or medical fastener 400 of the present invention. More specifically, the improved bone screw 400 is comprised of at least one smooth surface 402 and at least one textured surface 404. The screw component 400 is typically manufactured using additive manufacturing (AM) techniques, and the surface topography is grown as one integral part or unit.

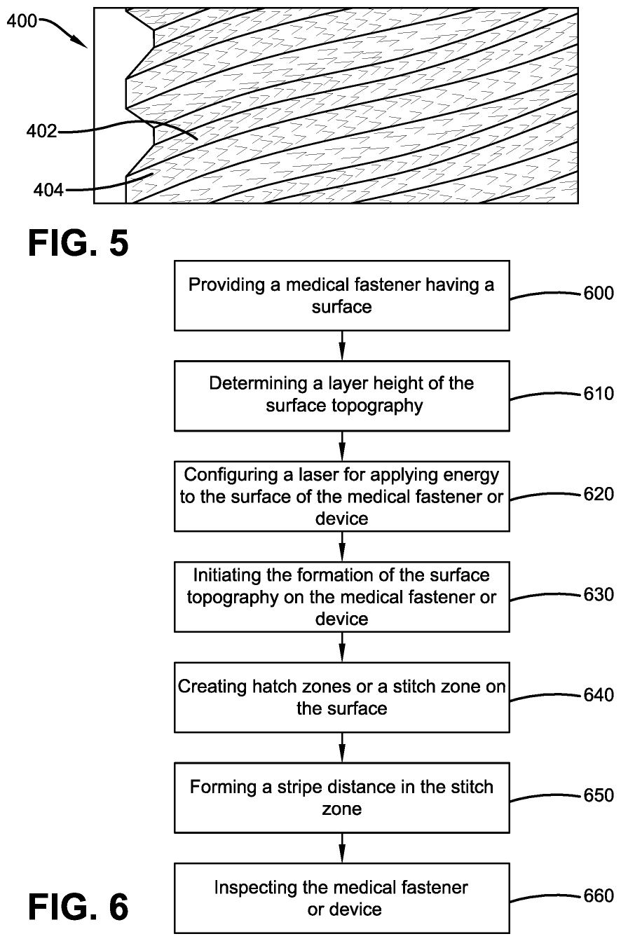

Additionally, the improved bone screw 400 and its various components may be of any suitable size, shape and/or configuration as is known in the art, without affecting the overall concept of the invention. One of ordinary skill in the art will appreciate that the shape, size and configuration of the bone screw or mechanical fastener 400 shown in FIGS. 4A-5 are for illustrative purposes only, and that many other shapes, sizes, and configurations of the bone screw or fastener 400 are well within the scope of the present disclosure. Although dimensions of the bone screw 400 (i.e., length, width, height, thread pitch, etc.) are important design parameters for good performance, the improved bone screw 400 may be of any shape, size, thread pitch or configuration that ensures optimal performance during use.

As shown in FIG. 4A, the improved bone screw 400 further comprises a cylindrical body 406 having a first end 408 and a closed second end 410. The cylindrical body 406 further includes a threaded portion 412 on an exterior surface portion 414 of the cylindrical body 406, a head 416 positioned at the first end 408 of cylindrical body 406 and configured to engage with a driver or other insertion tool (not shown) to advance the bone screw or fastener component 400 into a patient's bone (also not shown), and a penetrating or driving tip 418 located at the second end 410 of cylindrical body 406. The cylindrical body 406, the head 416 and the tip 418 are preferably formed as a single unit by additive manufacturing techniques, though it is contemplated that other manufacturing techniques could also be used without affecting the overall concept of the present invention.

Exterior thread 412 further comprises a surface 420. Surface 420 is has a smooth surface 402 and a controlled textured surface 404, wherein the controlled textured surface 404 exists in at least one direction and, preferably, only in one direction. More specifically, in a preferred embodiment of the present invention, the improved bone screw 400 comprises a smooth surface 402 in the positive, +/−X, +/−Y, and +Z directions of the exterior thread 414, and a textured surface 404 in only the −Z direction of the exterior thread 414. The presence of smooth surfaces 402 on the exterior thread 412 permits the improved bone screw 400 to be inserted into a patient's bone (not shown) relatively easily by minimizing the amount of insertion torque required during the installation. The smooth surface may also be provided with a frictionless coating, such as silicone, to increase the ease of inserting the screw into the bone. Additionally, the presence of the textured surface 404 in the negative direction (i.e., −Z) makes it more difficult for the improved bone screw 400 to come out, loosen or withdraw from the patient's bone prematurely, and promotes boney on-growth and mechanical interlocking. As shown in FIG. 4B, the medical fastener 400 may also be provided with an RFID chip 417 on the head so that information relating to the manufacturer of the screw, insertion date and other information may be contained on the chip 417 so that the chip may be read remotely from the body without the need for an invasive procedure to view the screw 400. The RFID chip 417 may include an insulating layer around the chip so as to lessen the interference between the chip and the metal in the bone screw when attempting to read the information contained on the chip 417.

FIG. 4C provides an alternate embodiment of the present invention showing a medical plate 450 having a surface topography 452 on only one side, namely the side that will be in contact with the bone of the patient. FIG. 4C also provides a bone screw 453 that has been inserted through the plate 450 which will be used to hold the plate over the fracture area of the bone.

FIG. 5 illustrates a perspective close-up view of the textured surface 404 on the exterior thread 412 of improved bone screw or medical fastener 400, as well as the smooth surface 402. The textured surface 404 can be any suitable shape as is known in the art, such as bumps, raises, nodules, ridges, helical, geometric configurations and combinations thereof, provided that it creates a raised, coarse or textured surface. Additionally, the textured surface 404 may be created via an additive manufacturing technique that is modified according to the laser parameters. More specifically, the amount of texture may be controlled by controlling the laser's operating parameters including, without limitation, the laser power, laser speed, and laser defocus.

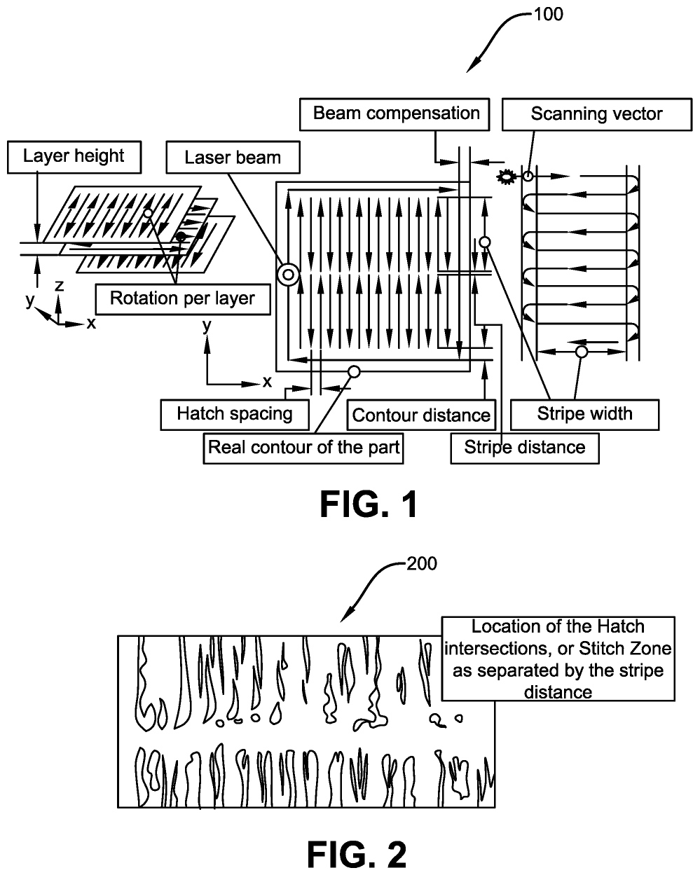

FIG. 1 illustrates a perspective view of the various machine parameters 100 that may be used to create a textured surface on one potential embodiment of the bone screw component 400 of the present invention in accordance with the disclosed architecture, wherein the machine parameters disclose the layer height and rotation per layer, as well as the hatch spacing, stripe width, and contour distance.

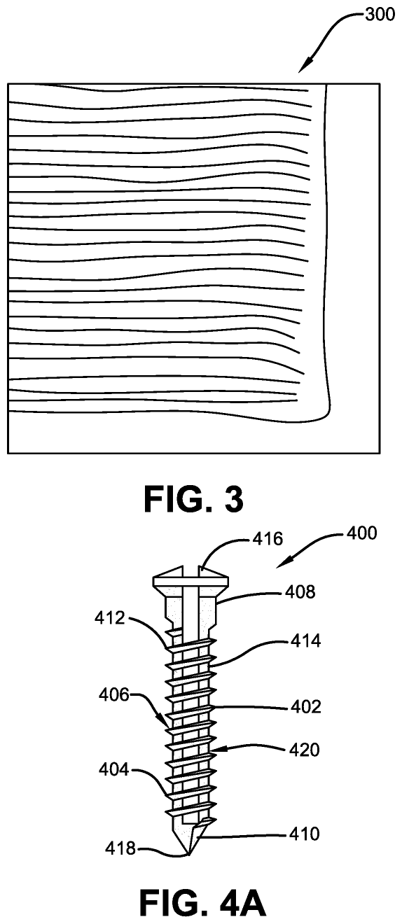

FIG. 2 illustrates a perspective view of the scan images 200 of the hatch intersections, as separated by the stripe distance on one potential embodiment of the bone screw component 400 of the present invention in accordance with the disclosed architecture, and FIG. 3 illustrates a perspective view of the scan images of the contour layers and hatch lines of one potential embodiment of the bone screw component of the present invention in accordance in accordance with the disclosed architecture.

FIG. 6 illustrates a block diagram of one potential embodiment of the method of manufacturing the bone screw component 400 of the present invention in accordance with the disclosed architecture. The method includes the step of initially providing a medical fastener or device at step 600, and then determining a height of the surface topography at step 610. At step 620, a laser is configured for applying energy to the surface of the medical fastener or device. Next, at step 630, the formation of the surface topography is initiated on the medical fastener or device. At step 640, hatch locations or a stitch zone is created on the surface of the device, and a stripe distance is formed in the stitch zone at step 650. At step 660, the device is inspected to insure that it conforms to the specifications.

Accordingly, while the improved bone screw is being driven in to a patient's bone, the trailing edge of the bone screw is pressing back against the bone to drive the screw component in place. Further, the presence of a relatively smooth surface 404 minimizes the amount of insertion torque required during the installation procedure, thereby also preventing or reducing the amount of user fatigue and/or the likelihood of bone fracture. Finally, the presence of the textured surface 404 in a single direction provides mechanical interlocking and greater potential for boney on-growth. The presence of the textured surface 404 in a single direction also reduces the likelihood that improved bone screw with prematurely back out or withdraw.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A medical fastener comprising:
a fastener having a first end, a second end and a body, the first end having a head and the second end having a penetrating tip; and
a surface topography created on a surface of the body, wherein the surface topography is provided in a single direction that is opposite that of an insertion direction.

2. The medical fastener as recited in claim 1, wherein the surface topography is formed from at least one of a bump, a raise, a nodule, a ridge, a helix, a geometric configuration or a combination thereof.

3. The medical fastener as recited in claim 1, wherein the single direction of the surface topography is provided in a positive direction.

4. The medical fastener as recited in claim 1, wherein the head is provided with an engagement feature for a driver or other insertion tool.

5. The medical fastener as recited in claim 1, wherein the body includes a threaded portion extending generally radially outward from the body.

6. The medical fastener as recited in claim 5, wherein the threaded portion has an upper surface and a lower surface.

7. The medical fastener as recited in claim 6, wherein the surface topography is provided on one of the upper and lower surfaces.

8. The medical fastener as recited in claim 7, wherein an insertion facilitating coating is provided on the body opposite to a surface topography coating.

9. The medical fastener as recited in claim 1, wherein the head comprises an RFID chip for receiving information about the medical fastener.

10. The medical fastener as recited in claim 8, wherein the surface topography coating has a stitch zone.

11. The medical fastener as recited in claim 10, wherein the stitch zone includes a stripe distance.

12. The medical fastener as recited in claim 2, wherein the surface topography is provided on a surface to engage a bone material.

13. A method of making a medical fastener comprising the steps of:
providing a fastener;
determining a height of a surface topography to be applied to a surface of the fastener;
configuring operational parameters of a laser;
applying energy from the laser to the surface of the fastener;
forming a topography on the surface;
creating a hatch location or a stitch zone on the surface; and
forming a stripe distance in the stitch zone.

14. The method of making a medical fastener as recited in claim 13, including a further step of inspecting the fastener after the step of forming.

15. The method of making a medical fastener as recited in claim 13, wherein the medical fastener is one of a screw, a rod or a pin.

16. The method of making a medical fastener as recited in claim 13, wherein the medical fastener has a first end having a head with an engagement feature and a second end with a penetration tip.

17. The method of making a medical fastener as recited in claim 13, wherein the medical fastener has a body with a threaded portion extending circumferentially around the body and extending radially outward from the body.

18. The method of making a medical fastener as recited in claim 13, wherein the step of forming is done by additive manufacturing.

19. The method of making a medical fastener as recited in claim 13, wherein the surface topography includes at least one of a bump, a raise, a nodule, a ridge, a helix, a geometric configuration or a combination thereof.

20. A bone screw comprising:
a body having a first end and a second end, with the first end having a head with an engagement feature for a driver or installation tool and the second end having a penetration tip; and
the body having a threaded portion between the first end and the second end, the threaded portion extending generally radially outwardly from the body and the threaded portion having an upper and lower surface, wherein a select one of the upper and lower surfaces having a texture applied thereto and further wherein the texture is applied in a single, bone facing direction.
